SEQUENCE LISTING (8 sequences)

SEQ ID NO: 1 - DNA, 1661 bp, Escherichia coli
  CDS (28)..(1629): aceB gene
ttcggatccatgacgaggagctgcacgatgactgaacaggcaacaacaaccgatgaactggctttcacaaggccgtatggcgagcaggagaagcaaattcttactgccgaagcggtagaatttctgactgagctggtgacgcattttacgccacaacgcaataaacttctggcagcgcgcattcagcagcagcaagatattgataacggaacgttgcctgattttatttcggaaacagcttccattcgcgatgctgattggaaaattcgcgggattcctgcggacttagaagaccgccgcgtagagataactggcccggtagagcgcaagatggtgatcaacgcgctcaacgccaatgtgaaagtctttatggccgatttcgaagattcactggcaccagactggaacaaagtgatcgacgggcaaattaacctgcgtgatgcggttaacggcaccatcagttacaccaatgaagcaggcaaaatttaccagctcaagcccaatccagcggttttgatttgtcgggtacgcggtctgcacttgccggaaaaacatgtcacctggcgtggtgaggcaatccccggcagcctgtttgattttgcgctctatttcttccacaactatcaggcactgttggcaaagggcagtggtccctatttctatctgccgaaaacccagtcctggcaggaagcggcctggtggagcgaagtcttcagctatgcagaagatcgctttaatctgccgcgcggcaccatcaaggcgacgttgctgattgaaacgctgcccgccgtgttccagatggatgaaatccttcacgcgctgcgtgaccatattgttggtctgaactgcggtcgttgggattacatcttcagctatatcaaaacgttgaaaaactatcccgatcgcgtcctgccagacagacaggcagtgacgatggataaaccattcctgaatgcttactcacgcctgttgattaaaacctgccataaacgcggtgcttttgcgatgggcggcatggcggcgtttattccgagcaaagatgaagagcacaataaccaggtgctcaacaaagtaaaagcggataaatcgctggaagccaataacggtcacgatggcacatggatcgctcacccaggccttgcggacacggcaatggcggtattcaacgacattctcggctcccgtaaaaatcagcttgaagtgatgcgcgaacaagacgcgccgattactgccgatcagctgctggcaccttgtgatggtgaacgcaccgaagaaggtatgcgcgccaacattcgcgtggctgtgcagtacatcgaagcgtggatctctggcaacggctgtgtgccgatttatggcctgatggaagatgcggcgacggctgaaatttcccgtacctcgatctggcagtggatccatcatcaaaaaacgttgagcaatggcaaaccggtgaccaaagccttgttccgccagatgctgggcgaagagatgaaagtcattgccagcgaactgggcgaagaacgtttctcccaggggcgttttgacgatgccgcacgcttgatggaacagatcaccacttccgatgagttaattgatttcctgaccctgccaggctaccgcctgttagcgtaaaccaccacataactatggagcatctgcacatg

SEQ ID NO: 2 - protein, 533 aa, Escherichia coli
MTEQATTTDELAFTRPYGEQEKQILTAEAVEFLTELVTHFTPQRNKLLAARIQQQQDIDNGTLPDFISETASIRDADWKIRGIPADLEDRRVEITGPVERKMVINALNANVKVFMADFEDSLAPDWNKVIDGQINLRDAVNGTISYTNEAGKIYQLKPNPAVLICRVRGLHLPEKHVTWRGEAIPGSLFDFALYFFHNYQALLAKGSGPYFYLPKTQSWQEAAWWSEVFSYAEDRFNLPRGTIKATLLIETLPAVFQMDEILHALRDHIVGLNCGRWDYIFSYIKTLKNYPDRVLPDRQAVTMDKPFLNAYSRLLIKTCHKRGAFAMGGMAAFIPSKDEEHNNQVLNKVKADKSLEANNGHDGTWIAHPGLADTAMAVFNDILGSRKNQLEVMREQDAPITADQLLAPCDGERTEEGMRANIRVAVQYIEAWISGNGCVPIYGLMEDAATAEISRTSIWQWIHHQKTLSNGKPVTKALFRQMLGEEMKVIASELGEERFSQGRFDDAARLMEQITTSDELIDFLTLPGYRLLA

SEQ ID NO: 3 - DNA, 20 bp, Artificial Sequence
  synthetic oligonucleotide
ttcggatccatgacgaggag

SEQ ID NO: 4 - DNA, 20 bp, Artificial Sequence
  synthetic oligonucleotide
ttgccaacagtgcctgatag

SEQ ID NO: 5 - DNA, 20 bp, Artificial Sequence
  synthetic oligonucleotide
atgcttactcacgcctgttg

SEQ ID NO: 6 - DNA, 20 bp, Artificial Sequence
  synthetic oligonucleotide
catgtgcagatgctccatag

SEQ ID NO: 7 - DNA, 1520 bp, Escherichia coli
  misc_feature (1)..(1520): mutagenic DNA
ctagatgcatgctcgagcggccgccagtgtgatggatatctgcagaattcggcttttcggatccatgacgaggagctgcacgatgactgaacaggcaacaacaaccgatgaactggctttcacaaggccgtatggcgagcaggagaagcaaattcttactgccgaagcggtagaatttctgactgagctggtgacgcattttacgccacaacgcaataaacttctggcagcgcgcattcagcagcagcaagatattgataacggaacgttgcctgattttatttcggaaacagcttccattcgcgatgctgattggaaaattcgcgggattcctgcggacttagaagaccgccgcgtagagataactggcccggtagagcgcaagatggtgatcaacgcgctcaacgccaatgtgaaagtctttatggccgatttcgaagattcactggcaccagactggaacaaagtgatcgacgggcaaattaacctgcgtgatgcggttaacggcaccatcagttacaccaatgaagcaggcaaaatttaccagctcaagcccaatccagcggttttgatttgtcgggtacgcggtctgcacttgccggaaaaacatgtcacctggcgtggtgaggcaatccccggcagcctgtttgattttgcgctctatttcttccacaactatcaggcactgttggcaaaagccgaattccagcacactggcggccgttactagtggatccgagatctgcagaattcggcttatgcttactcacgcctgttgattaaaacctgccataaacgcggtgcttttgcgatgggcggcatggcggcgtttattccgagcaaagatgaagagcacaataaccaggtgctcaacaaagtaaaagcggataaatcgctggaagccaataacggtcacgatggcacatggatcgctcacccaggccttgcggacacggcaatggcggtattcaacgacattctcggctcccgtaaaaatcagcttgaagtgatgcgcgaacaagacgcgccgattactgccgatcagctgctggcaccttgtgatggtgaacgcaccgaagaaggtatgcgcgccaacattcgcgtggctgtgcagtacatcgaagcgtggatctctggcaacggctgtgtgccgatttatggcctgatggaagatgcggcgacggctgaaatttcccgtacctcgatctggcagtggatccatcatcaaaaaacgttgagcaatggcaaaccggtgaccaaagccttgttccgccagatgctgggcgaagagatgaaagtcattgccagcgaactgggcgaagaacgtttctcccaggggcgttttgacgatgccgcacgcttgatggaacagatcaccacttccgatgagttaattgatttcctgaccctgccaggctaccgcctgttagcgtaaaccaccacataactatggagcatctgcacatgaagccgaattccagcacactggcggccgttactagtggatccgagctcggtac

SEQ ID NO: 8 - DNA, 1353 bp, Escherichia coli
  misc_feature (1)..(1353): mutagenic DNA
atgactgaacaggcaacaacaaccgatgaactggctttcacaaggccgtatggcgagcaggagaagcaaattcttactgccgaagcggtagaatttctgactgagctggtgacgcattttacgccacaacgcaataaacttctggcagcgcgcattcagcagcagcaagatattgataacggaacgttgcctgattttatttcggaaacagcttccattcgcgatgctgattggaaaattcgcgggattcctgcggacttagaagaccgccgcgtagagataactggcccggtagagcgcaagatggtgatcaacgcgctcaacgccaatgtgaaagtctttatggccgatttcgaagattcactggcaccagactggaacaaagtgatcgacgggcaaattaacctgcgtgatgcggttaacggcaccatcagttacaccaatgaagcaggcaaaatttaccagctcaagcccaatccagcggttttgatttgtcgggtacgcggtctgcacttgccggaaaaacatgtcacctggcgtggtgaggcaatccccggcagcctgtttgattttgcgctctatttcttccacaactatcaggcactgttggcaaaagccgaattccagcacactggcggccgttactagtggatccgagatctgcagaattcggcttatgcttactcacgcctgttgattaaaacctgccataaacgcggtgcttttgcgatgggcggcatggcggcgtttattccgagcaaagatgaagagcacaataaccaggtgctcaacaaagtaaaagcggataaatcgctggaagccaataacggtcacgatggcacatggatcgctcacccaggccttgcggacacggcaatggcggtattcaacgacattctcggctcccgtaaaaatcagcttgaagtgatgcgcgaacaagacgcgccgattactgccgatcagctgctggcaccttgtgatggtgaacgcaccgaagaaggtatgcgcgccaacattcgcgtggctgtgcagtacatcgaagcgtggatctctggcaacggctgtgtgccgatttatggcctgatggaagatgcggcgacggctgaaatttcccgtacctcgatctggcagtggatccatcatcaaaaaacgttgagcaatggcaaaccggtgaccaaagccttgttccgccagatgctgggcgaagagatgaaagtcattgccagcgaactgggcgaagaacgtttctcccaggggcgttttgacgatgccgcacgcttgatggaacagatcaccacttccgatgagttaattgatttcctgaccctgccaggctaccgcctgttagcgtaa

(19) United States
(12) Patent Application Publication
(10) Pub. No.: US 2005/0221448 A1
(43) Pub. Date: Oct. 6, 2005

(54) Process for the preparation of l-amino acids using strains of the enterobacteriaceae family which contain an attenuated aceb gene

(75) Inventor: Thomas Hermann, Bielefeld (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: DEGUSSA AG, Duesseldorf (DE)

(21) Appl. No.: 10/483,983

(22) PCT Filed: Jul. 3, 2002
(86) PCT No.: PCT/EP02/07352

§ 371 Date: Jan. 22, 2004

Related U.S. Application Data
(60) Provisional application No. 60/306,867, filed on Jul. 23, 2001.

(30) Foreign Application Priority Data
Jul. 18, 2001 (DE) 101 35 051.1

(51) Int. Cl. C12P 13/04; C12N 1/21

(52) U.S. Cl. 435/106; 435/252.33

(57) ABSTRACT

The invention relates to a process for the preparation of L-amino acids, in particular L-threonine, in which the following steps are carried out: a) fermentation of microorganisms of the Enterobacteriaceae family which produce the desired L-amino acid and in which the aceB gene, or the nucleotide sequence which codes for this, is attenuated, in particular eliminated, b) concentration of the L-amino acid in the medium or in the cells of the bacteria, and c) isolation of the L-amino acid.

### FIELD OF THE INVENTION

This invention relates to a process for the preparation of L-amino acids, in particular L-threonine, using strains of the Enterobacteriaceae family in which the aceB gene is attenuated.

### PRIOR ART

L-Amino acids, in particular L-threonine, are used in human medicine and in the pharmaceuticals industry, in the foodstuffs industry and very particularly in animal nutrition.

It is known to prepare L-amino acids by fermentation of strains of Enterobacteriaceae, in particular Escherichia coli (E. coli) and Serratia marcescens. Because of their great importance, work is constantly being undertaken to improve the preparation processes. Improvements to the process can relate to fermentation measures, such as e.g. stirring and supply of oxygen, or the composition of the nutrient media, such as e.g. the sugar concentration during the fermentation, or the working up to the product form, by e.g. ion exchange chromatography, or the intrinsic output properties of the microorganism itself.

Methods of mutagenesis, selection and mutant selection are used to improve the output properties of these microorganisms. Strains which are resistant to antimetabolites, such as e.g. the threonine analogue α-amino-β-hydroxyvaleric acid (AHV), or are auxotrophic for metabolites of regulatory importance and produce L-amino acid, such as e.g. L-threonine, are obtained in this manner.

Methods of the recombinant DNA technique have also been employed for some years for improving the strain of strains of the Enterobacteriaceae family which produce L-amino acids, by amplifying individual amino acid biosynthesis genes and investigating the effect on the production.

### OBJECT OF THE INVENTION

The object of the invention is to provide new measures for improved fermentative preparation of L-amino acids, in particular L-threonine.

### SUMMARY OF THE INVENTION

The invention provides a process for the fermentative preparation of L-amino acids, in particular L-threonine, using microorganisms of the Enterobacteriaceae family which in particular already produce L-amino acids and in which the nucleotide sequence which codes for the aceB gene is attenuated.

### DETAILED DESCRIPTION OF THE INVENTION

Where L-amino acids or amino acids are mentioned in the following, this means one or more amino acids, including their salts, chosen from the group consisting of L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-Threonine is particularly preferred.

The term “attenuation” in this connection describes the reduction or elimination of the intracellular activity of one or more enzymes (proteins) in a microorganism which are coded by the corresponding DNA, for example by using a weak promoter or a gene or allele which codes for a corresponding enzyme with a low activity or inactivates the corresponding enzyme (protein) or gene, and optionally combining these measures.

By attenuation measures, the activity or concentration of the corresponding protein is in general reduced to 0 to 75%, 0 to 50%, 0 to 25%, 0 to 10% or 0 to 5% of the activity or concentration of the wild-type protein or of the activity or concentration of the protein in the starting microorganism.

The process comprises carrying out the following steps:
a) fermentation of microorganisms of the Enterobacteriaceae family in which the aceB gene is attenuated,
b) concentration of the corresponding L-amino acid in the medium or in the cells of the microorganisms of the Enterobacteriaceae family, and
c) isolation of the desired L-amino acid, constituents of the fermentation broth and/or the biomass in its entirety or portions (>0 to 100%) thereof optionally remaining in the product.

The microorganisms which the present invention provides can produce L-amino acids from glucose, sucrose, lactose, fructose, maltose, molasses, optionally starch, optionally cellulose or from glycerol and ethanol. They are representatives of the Enterobacteriaceae family chosen from the genera Escherichia, Erwinia, Providencia and Serratia. The genera Escherichia and Serratia are preferred. Of the genus Escherichia the species Escherichia coli and of the genus Serratia the species Serratia marcescens are to be mentioned in particular.

Suitable strains, which produce L-threonine in particular, of the genus Escherichia, in particular of the species Escherichia coli, are, for example
Escherichia coli TF427
Escherichia coli H4578
Escherichia coli KY10935
Escherichia coli VNIIgenetika MG442
Escherichia coli VNIIgenetika M1
Escherichia coli VNIIgenetika 472T23
Escherichia coli BKIIM B-3996
Escherichia coli kat 13
Escherichia coli KCCM-10132

Suitable L-threonine-producing strains of the genus Serratia, in particular of the species Serratia marcescens, are, for example
Serratia marcescens HNr21
Serratia marcescens TLr156
Serratia marcescens T2000

Strains from the Enterobacteriaceae family which produce L-threonine preferably have, inter alia, one or more genetic or phenotypic features chosen from the group consisting of: resistance to α-amino-β-hydroxyvaleric acid, resistance to thialysine, resistance to ethionine, resistance to α-methylserine, resistance to diaminosuccinic acid, resistance to α-aminobutyric acid, resistance to borrelidin, resistance to rifampicin, resistance to valine analogues, such as, for example, valine hydroxamate, resistance to purine analogues, such as, for example, 6-dimethylaminopurine, a need for L-methionine, optionally a partial and compensable need for L-isoleucine, a need for meso-diaminopimelic acid, auxotrophy in respect of threonine-containing dipeptides, resistance to L-threonine, resistance to L-homoserine, resistance to L-lysine, resistance to L-methionine, resistance to L-glutamic acid, resistance to L-aspartate, resistance to L-leucine, resistance to L-phenylalanine, resistance to L-serine, resistance to L-cysteine, resistance to L-valine, sensitivity to fluoropyruvate, defective threonine dehydrogenase, optionally an ability for sucrose utilization, enhancement of the threonine operon, enhancement of homoserine dehydrogenase I-aspartate kinase I, preferably of the feed back resistant form, enhancement of homoserine kinase, enhancement of threonine synthase, enhancement of aspartate kinase, optionally of the feed back resistant form, enhancement of aspartate semialdehyde dehydrogenase, enhancement of phosphoenol pyruvate carboxylase, optionally of the feed back resistant form, enhancement of phosphoenol pyruvate synthase, enhancement of transhydrogenase, enhancement of the RhtB gene product, enhancement of the RhtC gene product, enhancement of the YfiK gene product, enhancement of a pyruvate carboxylase, and attenuation of acetic acid formation.

It has been found that microorganisms of the Enterobacteriaceae family produce L-amino acids, in particular L-threonine, in an improved manner after attenuation, in particular elimination, of the aceB gene.

The nucleotide sequences of the genes of Escherichia coli belong to the prior art and can also be found in the genome sequence of Escherichia coli published by Blattner et al. (Science 277: 1453-1462 (1997)).

The aceB gene is described, inter alia, by the following data:
Description: Malate synthase A
EC No.: 4.1.3.2
Reference: Byrne et al.; Nucleic Acids Research 16(19), 9342 (1988); Byrne et al.; Nucleic Acids Research 16(22), 10924 (1988); Cortay et al.; Biochimie 71(9-10): 1043-9 (1989)
Accession No.: AE000474
Alternative gene name: mas

The nucleic acid sequences can be found in the databanks of the National Center for Biotechnology Information (NCBI) of the National Library of Medicine (Bethesda, Md., USA), the nucleotide sequence databank of the European Molecular Biologies Laboratories (EMBL, Heidelberg, Germany or Cambridge, UK) or the DNA databank of Japan (DDBJ, Mishima, Japan).

The genes described in the text references mentioned can be used according to the invention. Alleles of the genes which result from the degeneracy of the genetic code or due to “sense mutations” of neutral function can furthermore be used.

To achieve an attenuation, for example, expression of the gene or the catalytic properties of the enzyme proteins can be reduced or eliminated. The two measures can optionally be combined.

The reduction in gene expression can take place by suitable culturing, by genetic modification (mutation) of the signal structures of gene expression or also by the antisense-RNA technique. Signal structures of gene expression are, for example, repressor genes, activator genes, operators, promoters, attenuators, ribosome binding sites, the start codon and terminators. The expert can find information in this respect, inter alia, for example, in Jensen and Hammer (Biotechnology and Bioengineering 58: 191-195 (1998)), in Carrier and Keasling (Biotechnology Progress 15: 58-64 (1999), Franch and Gerdes (Current Opinion in Microbiology 3: 159-164 (2000)) and in known textbooks of genetics and molecular biology, such as, for example, the textbook of Knippers (“Molekulare Genetik [Molecular Genetics]”, 6th edition, Georg Thieme Verlag, Stuttgart, Germany, 1995) or that of Winnacker (“Gene und Klone [Genes and Clones]”, VCH Verlagsgesellschaft, Weinheim, Germany, 1990).

Mutations which lead to a change or reduction in the catalytic properties of enzyme proteins are known from the prior art. Examples which may be mentioned are the works of Qiu and Goodman (Journal of Biological Chemistry 272: 8611-8617 (1997)), Yano et al. (Proceedings of the National Academy of Sciences of the United States of America 95: 5511-5515 (1998), Wente and Schachmann (Journal of Biological Chemistry 266: 20833-20839 (1991)). Summarizing descriptions can be found in known textbooks of genetics and molecular biology, such as e.g. that by Hagemann (“Allgemeine Genetik [General Genetics]”, Gustav Fischer Verlag, Stuttgart, 1986).

Possible mutations are transitions, transversions, insertions and deletions. Depending on the effect of the amino acid exchange on the enzyme activity, “missense mutations” or “nonsense mutations” are referred to. Insertions or deletions of at least one base pair in a gene lead to “frame shift mutations”, which lead to incorrect amino acids being incorporated or translation being interrupted prematurely. If a stop codon is formed in the coding region as a consequence of the mutation, this also leads to a premature termination of the translation. Deletions of several codons typically lead to a complete loss of the enzyme activity. Instructions on generation of such mutations are prior art and can be found in known textbooks of genetics and molecular biology, such as e.g. the textbook by Knippers (“Molekulare Genetik [Molecular Genetics]”, 6th edition, Georg Thieme Verlag, Stuttgart, Germany, 1995), that by Winnacker (“Gene und Klone [Genes and Clones]”, VCH Verlagsgesellschaft, Weinheim, Germany, 1990) or that by Hagemann (“Allgemeine Genetik [General Genetics]”, Gustav Fischer Verlag, Stuttgart, 1986).

Suitable mutations in the genes, such as, for example, deletion mutations, can be incorporated into suitable strains by gene or allele replacement.

A conventional method is the method, described by Hamilton et al. (Journal of Bacteriology 171: 4617-4622 (1989)), of gene replacement with the aid of a conditionally replicating pSC101 derivative pMAK705. Other methods described in the prior art, such as, for example, those of Martinez-Morales et al. (Journal of Bacteriology 181: 7143-7148 (1999)) or those of Boyd et al. (Journal of Bacteriology 182: 842-847 (2000)), can likewise be used.

It is also possible to transfer mutations in the particular genes or mutations which affect expression of the particular genes into various strains by conjugation or transduction.

It may furthermore be advantageous for the production of L-amino acids, in particular L-threonine, with strains of the Enterobacteriaceae family, in addition to attenuation of the aceB gene, for one or more enzymes of the known threonine biosynthesis pathway or enzymes of anaplerotic metabolism or enzymes for the production of reduced nicotinamide adenine dinucleotide phosphate or enzymes of glycolysis or PTS enzymes or enzymes of sulfur metabolism to be enhanced.

The term “enhancement” in this connection describes the increase in the intracellular activity of one or more enzymes or proteins in a microorganism which are coded by the corresponding DNA, for example by increasing the number of copies of the gene or genes, using a potent promoter or a gene which codes for a corresponding enzyme or protein with a high activity, and optionally combining these measures.

By enhancement measures, in particular over-expression, the activity or concentration of the corresponding protein is in general increased by at least 10%, 25%, 50%, 75%, 100%, 150%, 200%, 300%, 400% or 500%, up to a maximum of 1000% or 2000%, based on that of the wild-type protein or the activity or concentration of the protein in the starting microorganism.

Thus, for example, at the same time one or more of the genes chosen from the group consisting of
the thrABC operon which codes for aspartate kinase, homoserine dehydrogenase, homoserine kinase and threonine synthase (U.S. Pat. No. 4,278,765),
the pyc gene of Corynebacterium glutamicum which codes for pyruvate carboxylase (WO 99/18228),
the pps gene which codes for phosphoenol pyruvate synthase (Molecular and General Genetics 231 (2): 332-336 (1992)),
the ppc gene which codes for phosphoenol pyruvate carboxylase (Gene 31: 279-283 (1984)),
the pntA and pntB genes which code for transhydrogenase (European Journal of Biochemistry 158: 647-653 (1986)),
the rhtB gene which imparts homoserine resistance (EP-A-0 994 190),
the mqo gene which codes for malate:quinone oxidoreductase (WO 02/06459),
the rhtC gene which imparts threonine resistance (EP-A-1 013 765),
the thrE gene of Corynebacterium glutamicum which codes for the threonine export protein (WO 01/92545),
the gdhA gene which codes for glutamate dehydrogenase (Nucleic Acids Research 11: 5257-5266 (1983); Gene 23: 199-209 (1983)),
the hns gene which codes for the DNA-binding protein HLP-II (Molecular and General Genetics 212: 199-202 (1988)),
the pgm gene which codes for phosphoglucomutase (Journal of Bacteriology 176: 5847-5851 (1994)),
the fba gene which codes for fructose biphosphate aldolase (Biochemical Journal 257: 529-534 (1989)),
the ptsH gene of the ptsHIcrr operon which codes for the phosphohistidine protein hexose phosphotransferase of the phosphotransferase system PTS (Journal of Biological Chemistry 262: 16241-16253 (1987)),
the ptsI gene of the ptsHIcrr operon which codes for enzyme I of the phosphotransferase system PTS (Journal of Biological Chemistry 262: 16241-16253 (1987)),
the crr gene of the ptsHIcrr operon which codes for the glucose-specific IIA component of the phosphotransferase system PTS (Journal of Biological Chemistry 262: 16241-16253. (1987)),
the ptsG gene which codes for the glucose-specific IIBC component (Journal of Biological Chemistry 261: 16398-16403 (1986)),
the lrp gene which codes for the regulator of the leucine regulon (Journal of Biological Chemistry 266: 10768-10774 (1991)),
the mopB gene which codes for 10 Kd chaperone (Journal of Biological Chemistry 261: 12414-12419 (1986)) and is also known by the name groES,
the ahpc gene of the ahpCF operon which codes for the small sub-unit of alkyl hydroperoxide reductase (Proceedings of the National Academy of Sciences of the United States of America 92: 7617-7621 (1995)),
the ahpF gene of the ahpCF operon which codes for the large sub-unit of alkyl hydroperoxide reductase (Proceedings of the National Academy of Sciences of the United States of America 92: 7617-7621 (1995)),
the cysK gene which codes for cysteine synthase A (Journal of Bacteriology 170: 3150-3157 (1988)),
the cysB gene which codes for the regulator of the cys regulon (Journal of Biological Chemistry 262: 5999-6005 (1987)),
the cysJ gene of the cysJIH operon which codes for the flavoprotein of NADPH sulfite reductase (Journal of Biological Chemistry 264: 15796-15808 (1989), Journal of Biological Chemistry 264: 15726-15737 (1989)),
the cysI gene of the cysJIH operon which codes for the haemoprotein of NADPH sulfite reductase (Journal of Biological Chemistry 264: 15796-15808 (1989), Journal of Biological Chemistry 264: 15726-15737 (1989)) and
the cysH gene of the cysJIH operon which codes for adenylyl sulfate reductase (Journal of Biological Chemistry 264: 15796-15808 (1989), Journal of Biological Chemistry 264: 15726-15737 (1989))
   can be enhanced, in particular over-expressed.

The use of endogenous genes is in general preferred. “Endogenous genes” or “endogenous nucleotide sequences” are understood as meaning the genes or nucleotide sequences present in the population of a species.

It may furthermore be advantageous for the production of L-amino acids, in particular L-threonine, in addition to attenuation of the aceB gene, for one or more of the genes chosen from the group consisting of
the tdh gene which codes for threonine dehydrogenase (Journal of Bacteriology 169: 4716-4721 (1987)),
the mdh gene which codes for malate dehydrogenase (E.C. 1.1.1.37) (Archives in Microbiology 149: 36-42 (1987)),
the gene product of the open reading frame (orf) yjfA (Accession Number AAC77180 of the National Center for Biotechnology Information (NCBI, Bethesda, Md., USA)),
the gene product of the open reading frame (orf) ytfP (Accession Number AAC77179 of the National Center for Biotechnology Information (NCBI, Bethesda, Md., USA)),
the pckA gene which codes for the enzyme phosphoenol pyruvate carboxykinase (Journal of Bacteriology 172: 7151-7156 (1990)),
the poxB gene which codes for pyruvate oxidase (Nucleic Acids Research 14 (13): 5449-5460 (1986)),
the aceA gene which codes for the enzyme isocitrate lyase (Journal of Bacteriology 170: 4528-4536 (1988)),
the dgsA gene which codes for the DgsA regulator of the phosphotransferase system (Bioscience, Biotechnology and Biochemistry 59: 256-261 (1995)) and is also known under the name of the m1c gene,
the fruR gene which codes for the fructose repressor (Molecular and General Genetics 226: 332-336 (1991)) and is also known under the name of the cra gene,
the rpoS gene which codes for the sigma³⁸ factor (WO 01/05939) and is also known under the name of the katF gene,
the aspA gene which codes for aspartate ammonium lyase (Nucleic Acids Research 13 (6): 2063-2074 (1985)),
the aceK gene which codes for isocitrate dehydrogenase kinase/phosphatase (Journal of Bacteriology 170 (1): 89-97 (1988)) and
the ugpB gene which codes for the periplasmic binding protein of the sn-glycerol 3-phosphate transport system (Molecular Microbiology 2 (6): 767-775 (1988))
   to be attenuated, in particular eliminated or for the expression thereof to be reduced.

It may furthermore be advantageous for the production of L-amino acids, in particular L-threonine, in addition to attenuation of the aceB gene, to eliminate undesirable side reactions (Nakayama: “Breeding of Amino Acid Producing Microorganisms”, in: Overproduction of Microbial Products, Krumphanzl, Sikyta, Vanek (eds.), Academic Press, London, UK, 1982).

The microorganisms produced according to the invention can be cultured in the batch process (batch culture), the fed batch process (feed process) or the repeated fed batch process (repetitive feed process). A summary of known culture methods is described in the textbook by Chmiel (Bioprozesstechnik 1. Einführung in die Bioverfahrenstechnik [Bioprocess Technology 1]. Introduction to Bioprocess Technology (Gustav Fischer Verlag, Stuttgart, 1991)) or in the textbook by Storhas (Bioreaktoren und periphere Einrichtungen [Bioreactors and Peripheral Equipment] (Vieweg Verlag, Braunschweig/Wiesbaden, 1994)).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981).

Sugars and carbohydrates, such as e.g. glucose, sucrose, lactose, fructose, maltose, molasses, starch and optionally cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and ethanol, and organic acids, such as e.g. acetic acid, can be used as the source of carbon. These substances can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Phosphoric acid, potassium dihydrogen phosphate or dipotassium hydrogen phosphate or the corresponding sodium-containing salts can be used as the source of phosphorus. The culture medium must furthermore comprise salts of metals, such as e.g. magnesium sulfate or iron sulfate, which are necessary for growth. Finally, essential growth substances, such as amino acids and vitamins, can be employed in addition to the abovementioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia, or acid compounds, such as phosphoric acid or sulfuric acid, can be employed in a suitable manner to control the pH of the culture. Antifoams, such as e.g. fatty acid polyglycol esters, can be employed to control the development of foam. Suitable substances having a selective action, e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 25° C. to 45° C., and preferably 30° C. to 40° C. Culturing is continued until a maximum of L-amino acids or L-threonine has formed. This target is usually reached within 10 hours to 160 hours.

The analysis of L-amino acids can be carried out by anion exchange chromatography with subsequent ninhydrin derivation, as described by Spackman et al. (Analytical Chemistry 30: 1190-1206 (1958)), or it can take place by reversed phase HPLC as described by Lindroth et al. (Analytical Chemistry 51: 1167-1174 (1979)).

The process according to the invention is used for the fermentative preparation of L-amino acids, such as, for example, L-threonine, L-isoleucine, L-valine, L-methionine, L-homoserine and L-lysine, in particular L-threonine.

A pure culture of the Escherichia coli K-12 strain DH5α/pMAK705 was deposited as DSM 13720 on 8 Sep. 2000 at the Deutsche Sammlung für Mikroorganismen und Zellkulturen (DSMZ=German Collection of Microorganisms and Cell Cultures, Braunschweig, Germany) in accordance with the Budapest Treaty.

The present invention is explained in more detail in the following with the aid of embodiment examples.

The isolation of plasmid DNA from Escherichia coli and all techniques of restriction, ligation, Klenow and alkaline phosphatase treatment are carried out by the method of Sambrook et al. (Molecular Cloning—A Laboratory Manual (1989) Cold Spring Harbor Laboratory Press). Unless described otherwise, the transformation of Escherichia coli is carried out by the method of Chung et al. (Proceedings of the National Academy of Sciences of the United States of America 86: 2172-2175 (1989)).

The incubation temperature for the preparation of strains and transformants is 37° C. Temperatures of 30° C. and 44° C. are used in the gene replacement method of Hamilton et al.

### EXAMPLE 1

Construction of the Deletion Mutation of the aceB Gene

Parts of the gene regions lying upstream and downstream of the aceB gene and parts of the 5′ and 3′ region of the aceB gene are amplified from Escherichia coli K12 using the polymerase chain reaction (PCR) and synthetic oligonucleotides. Starting from the nucleotide sequence of the aceB gene and sequences lying upstream and downstream in E. coli K12 MG1655 (SEQ ID No. 1, Accession No. AE000474), the following PCR primers are synthesized (MWG Biotech, Ebersberg, Germany):

| | |
|---|---|
| aceB5′-1: | |
| 5′ - TTCGGATCCATGACGAGGAG - 3′ | (SEQ ID No. 3) |
| aceB5′-2: | |
| 5′ - TTGCCAACAGTGCCTGATAG - 3′ | (SEQ ID No. 4) |
| aceB3′-1: | |
| 5′ - ATGCTTACTCACGCCTGTTG - 3′ | (SEQ ID No. 5) |
| aceB3′-2: | |
| 5′ - CATGTGCAGATGCTCCATAG - 3′ | (SEQ ID No. 6) |

The chromosomal E. coli K12 MG1655 DNA employed for the PCR is isolated according to the manufacturer's instructions with “Qiagen Genomic-tips 100/G” (QIAGEN, Hilden, Germany). A DNA fragment approx. 650 bp in size from the 5′ region of the aceB gene region (called aceB5′) and a DNA fragment approx. 700 bp in size from the 3′ region of the aceB gene region (called aceB3′) can be amplified with the specific primers under standard PCR conditions (Innis et al. (1990) PCR Protocols. A Guide to Methods and Applications, Academic Press) with Taq-DNA polymerase (Gibco-BRL, Eggenstein, Germany). The PCR products are each ligated with the vector pCRII-TOPO (TOPO TA Cloning Kit, Invitrogen, Groningen, The Netherlands) in accordance with the manufacturer's instructions and transformed into the E. coli strain TOP10F′. Selection of plasmid-carrying cells takes place on LB agar, to which 50 μg/ml ampicillin are added. After isolation of the plasmid DNA, the vector pCRII-TOPOaceB3′, is cleaved with the restriction enzymes XbaI and Ec1136II. The aceB5′, fragment is isolated after separation in 0.8% agarose gel with the aid of the QIAquick Gel Extraction Kit (QIAGEN, Hilden, Germany). After isolation of the plasmid DNA the vector pCRII-TOPOaceB5′ is cleaved with the enzymes EcoRV and XbaI and ligated with the aceB5′ fragment isolated. The E. coli strain DH5α is transformed with the ligation batch and plasmid-carrying cells are selected on LB agar, to which 50 μg/ml ampicillin are added. After isolation of the plasmid DNA those plasmids in which the mutagenic DNA sequence shown in SEQ ID No. 7 is cloned are detected by control cleavage with the enzymes Bc1I, HincII, SpeI and SphI. One of the plasmids is called pCRII-TOPOΔaceB (=pCRII-TOPOdeltaaceB).

### EXAMPLE 2

Construction of the Replacement Vector pMAK705ΔaceB

The ΔaceB allele described in example 1 is isolated from the vector pCRII-TOPOΔaceB after restriction with the enzymes KpnI and XbaI and separation in 0.8% agarose gel, and ligated with the plasmid pMAK705 (Hamilton et al., Journal of Bacteriology 171: 4617-4622 (1989)), which has been digested with the enzymes KpnI and XbaI. The ligation batch is transformed in DH5α and plasmid-carrying cells are selected on LB agar, to which 20 μg/ml chloramphenicol are added. Successful cloning is demonstrated after isolation of the plasmid DNA and cleavage with the enzymes BamHI, ClaI, PauI and XbaI. The replacement vector formed, pMAK705ΔaceB (=pMAK705deltaaceB), is shown in FIG. 1.

### EXAMPLE 3

Position-Specific Mutagenesis of the aceB Gene in the E. Coli Strain MG442

The L-threonine-producing E. coli strain MG442 is described in the patent specification U.S. Pat. No. 4,278,765 and deposited as CMIM B-1628 at the Russian National Collection for Industrial Microorganisms (VKPM, Moscow, Russia).

For replacement of the chromosomal aceB gene with the plasmid-coded deletion construct, MG442 is transformed with the plasmid pMAK705ΔaceB. The gene replacement is carried out using the selection method described by Hamilton et al. (Journal of Bacteriology 171: 4617-4622 (1989)) and is verified by standard PCR methods (Innis et al. (1990) PCR Protocols. A Guide to Methods and Applications, Academic Press) with the following oligonucleotide primers:

| | |
|---|---|
| aceB5′-1: | |
| 5′ - TTCGGATCCATGACGAGGAG - 3′ | (SEQ ID No. 3) |
| aceB3′-2: | |
| 5′ - CATGTGCAGATGCTCCATAG - 3′ | (SEQ ID No. 6) |

After replacement has taken place, MG442 contains the form of the ΔaceB allele shown in SEQ ID No. 8. The strain obtained is called MG442ΔaceB.

### EXAMPLE 4

Preparation of L-Threonine with the Strain MG442ΔaceB

MG442ΔaceB is multiplied on minimal medium with the following composition: 3.5 g/l Na₂HPO₄*2H₂O, 1.5 g/l KH₂PO₄, 1 g/l NH₄Cl, 0.1 g/l MgSO₄*7H₂O, 2 g/l glucose, 20 g/l agar. The formation of L-threonine is checked in batch cultures of 10 ml contained in 100 ml conical flasks. For this, 10 ml of preculture medium of the following composition: 2 g/l yeast extract, 10 g/l (NH₄)₂SO₄, 1 g/l KH₂PO₄, 0.5 g/l MgSO₄*7H₂O, 15 g/l CaCO₃, 20 g/l glucose are inoculated and the batch is incubated for 16 hours at 37° C. and 180 rpm on an ESR incubator from Kümer AG (Birsfelden, Switzerland). 250 μl of this preculture are transinoculated into 10 ml of production medium (25 g/l (NH₄)₂SO₄, 2 g/l KH₂PO₄, 1 μl MgSO₄*7H₂O, 0.03 g/l FeSO₄*7H₂O, 0.018 g/l MnSO₄*1H₂O, 30 g/l CaCO₃, 20 g/l glucose) and the batch is incubated for 48 hours at 37° C. After the incubation the optical density (OD) of the culture suspension is determined with an LP2W photometer from Dr. Lange (Düsseldorf, Germany) at a measurement wavelength of 660 nm.

The concentration of L-threonine formed is then determined in the sterile-filtered culture supernatant with an amino acid analyzer from Eppendorf-BioTronik (Hamburg, Germany) by ion exchange chromatography and post-column reaction with ninhydrin detection.

The result of the experiment is shown in Table 1.

TABLE 1

| | OD | L-Threonine |
|---|---|---|
| Strain | (660 nm) | g/l |
| MG442 | 6.0 | 1.5 |
| MG442ΔaceB | 4.8 | 2.1 |

### BRIEF DESCRIPTION OF THE FIGURES

FIG. 1: pMAK705ΔaceB (=pMAK705deltaaceB)

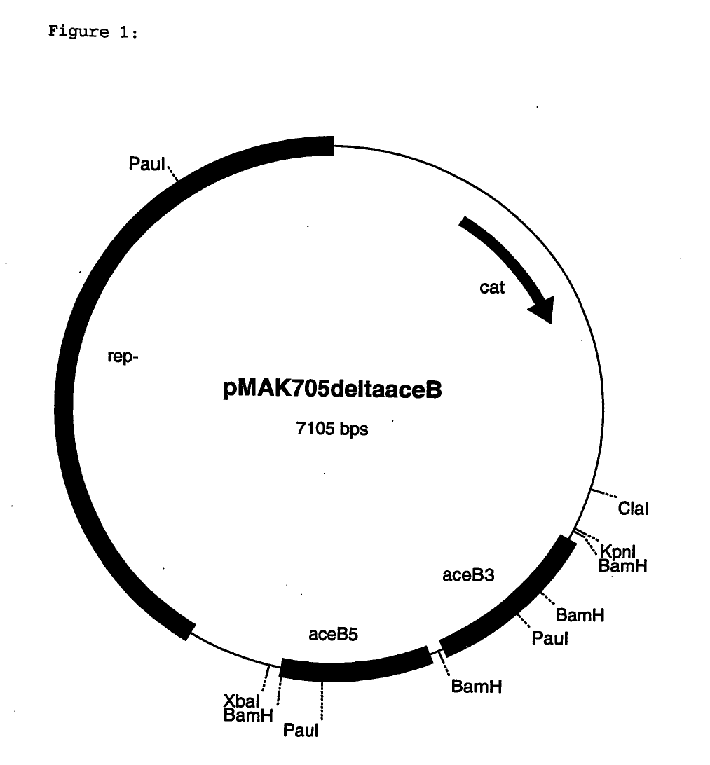

The length data are to be understood as approx. data. The abbreviations and designations used have the following meaning:
cat: Chloramphenicol resistance gene
rep-ts: Temperature-sensitive replication region of the plasmid pSC101
aceB5′: Part of the 5′ region of the aceB gene and the region lying upstream
aceB3′: Part of the 3′ region of the aceB gene and the region lying downstream

The abbreviations for the restriction enzymes have the following meaning
BamHI: Restriction endonuclease from Bacillus amyloliquefaciens H
ClaI: Restriction endonuclease from Caryphanon latum
KpnI: Restriction endonuclease from Klebsiella pneumoniae
PauI: Restriction endonuclease from Paracoccus alcaliphilus
XbaI: Restriction endonuclease from Xanthomonas badrii

1. A process for the preparation of L-amino acids, in particular L-threonine, which comprises carrying out the following steps:
a) fermentation of microorganisms of the Enterobacteriaceae family which produce the desired L-amino acid and in which the aceB gene, or the nucleotide sequence which codes for this, is attenuated, in particular eliminated,
b) concentration of the desired L-amino acid in the medium or in the cells of the microorganisms, and
c) isolation of the desired L-amino acid, constituents of the fermentation broth and/or the biomass in its entirety or portions (>0 to 100%) thereof optionally remaining in the product.

2. A process as claimed in claim 1, wherein microorganisms in which further genes of the biosynthesis pathway of the desired L-amino acid are additionally enhanced are employed.

3. A process as claimed in claim 1, wherein microorganisms in which the metabolic pathways which reduce the formation of the desired L-amino acid are at least partly eliminated are employed.

4. A process as claimed in claim 1, wherein the expression of the polynucleotide which codes for the aceB gene is attenuated, in particular eliminated.

5. A process as claimed in claim 1, wherein the regulatory and/or catalytic properties of the polypeptide (enzyme protein) for which the polynucleotide aceB codes are reduced.

6. A process as claimed in claim 1, wherein, for the preparation of L-amino acids, microorganisms of the Enterobacteriaceae family in which in addition at the same time one or more of the genes chosen from the group consisting of:
6.1 the thrABC operon which codes for aspartate kinase, homoserine dehydrogenase, homoserine kinase and threonine synthase,
6.2 the pyc gene which codes for pyruvate carboxylase,
6.3 the pps gene which codes for phosphoenol pyruvate synthase,
6.4 the ppc gene which codes for phosphoenol pyruvate carboxylase,
6.5 the pntA and pntB genes which code for transhydrogenase,
6.6 the rhtB gene which imparts homoserine resistance,
6.7 the mqo gene which codes for malate:guinone oxidoreductase,
6.8 the rhtC gene which imparts threonine resistance,
6.9 the thrE gene which codes for the threonine export protein,
6.10 the gdhA gene which codes for glutamate dehydrogenase,
6.11 the hns gene which codes for the DNA-binding protein HLP-II,
6.12 the pgm gene which codes for phosphoglucomutase,
6.13 the fba gene which codes for fructose biphosphate aldolase,
6.14 the ptsH gene which codes for the phosphohistidine protein hexose phosphotransferase,
6.15 the ptsI gene which codes for enzyme I of the phosphotransferase system,
6.16 the crr gene which codes for the glucose-specific IIA component,
6.17 the ptsG gene which codes for the glucose-specific IIBC component,
6.18 the lrp gene which codes for the regulator of the leucine regulon,
6.19 the mopB gene which codes for 10 Kd chaperone,
6.20 the ahpC gene which codes for the small sub-unit of alkyl hydroperoxide reductase,
6.21 the ahpF gene which codes for the large sub-unit of alkyl hydroperoxide reductase,
6.22 the cysK gene which codes for cysteine synthase A,
6.23 the cysB gene which codes for the regulator of the cys regulon,
6.24 the cysJ gene which codes for the flavoprotein of NADPH sulfite reductase,
6.25 the cysI gene which codes for the haemoprotein of NADPH sulfite reductase and
6.26 the cysH gene which codes for adenylyl sulfate reductase,
is or are enhanced, in particular over-expressed, are fermented.

7. A process as claimed in claim 1, wherein, for the preparation of L-amino acids, microorganisms of the Enterobacteriaceae family in which in addition at the same time one or more of the genes chosen from the group consisting of:
7.1 the tdh gene which codes for threonine dehydrogenase,
7.2 the mdh gene which codes for malate dehydrogenase,
7.3 the gene product of the open reading frame (orf) yjfA,
7.4 the gene product of the open reading frame (orf) ytfP,
7.5 the pcka gene which codes for phosphoenol pyruvate carboxykinase
7.6 the poxB gene which codes for pyruvate oxidase
7.7 the aceA gene which codes for isocitrate lyase,
7.8 the dgsA gene which codes for the DgsA regulator of the phosphotransferase system,
7.9 the fruR gene which codes for the fructose repressor,
7.10 the rpoS gene which codes for the sigma³⁸ factor,
7.11 the aspA gene which codes for aspartate ammonium lyase,
7.12 the aceK gene which codes for isocitrate dehydrogenase kinase/phosphatase and
7.13 the ugpB gene which codes for the periplasmic binding protein of the sn-glycerol 3-phosphate transport system
is or are attenuated, in particular eliminated or reduced in expression, are fermented.
